(19) United States
(12) Patent Application Publication
(10) Pub. No.: US 2006/0147535 A1
(43) Pub. Date: Jul. 6, 2006

(54) Methods for and compositions of anticancer medicaments

(76) Inventors: Poongunran Muthukumaran, Worcester, MA (US); Lalit Chordia, Pittsburgh, PA (US)

Correspondence Address: POONGUNRAN MUTHUKUMARAN;THAR TECHNOLOGIES<INC, 730 WILLIAM PITT WAY, PITTSBURGH, PA, 15238, US

(21) Appl. No.: 10/825,963

(22) Filed: Nov. 24, 2004

Related U.S. Application Data
(60) Provisional application No. 60/463,445, filed on Apr. 16, 2003.

(51) Int. Cl. A61K 9/14

(52) U.S. Cl. 424/489; 977/906

(57) ABSTRACT

The present invention provides methods for and compositions of anticancer medicaments. These compositions are comprised of nanoparticles or microparticles produced by antisolvent technology. The particles can be used to treat cancerous tissues in humans or animals.

### CROSS-REFERENCE TO RELATED PATENT APPLICATIONS

This patent application claims the benefit of U.S. Provisional Patent Application No. 60/463,445 filed Apr. 16, 2003 titled Methods for and Compositions of Anticancer Medicaments.

### BACKGROUND OF THE INVENTION

1. Field of Invention

The current invention relates to methods for and compositions of anticancer medicaments. Methods include producing nanoparticles and microparticles using antisolvent technology. The invention provides compositions of anticancer medicaments to be used in human or animal treatment of cancerous tissues.

2. Background

The formation of fine particles of desired substances in the micro- to nanometer range is an intense area of research. The processes and methods can be extended to a wide variety of materials, including catalysts, chemicals, coatings, explosives, pesticides, polymers and pharmaceuticals. Many supercritical fluid processes have been used to produce fine particles. Most of the research has focused on using either the supercritical fluid as a solvent or an antisolvent. In the Rapid Expansion of Supercritical Solutions (RESS) process, the supercritical fluid is used as the solvent, whereas in Supercritical antisolvent processes (SAS) processes the supercritical fluid is used an antisolvent. The choice of the process depends on the solubility of the material of interest in the supercritical fluid. Some examples of the particles formed using these techniques include steroids (Larson and King, 1985), polystyrene (Dixon et al., 1993), trypsin (Winter et al., 1993) and insulin (Yeo et al., 1993; Winter et al., 1993). Other work has focused on the formation of fine polymeric particles that contain various drugs for the purpose of controlled drug release (Tom et al., 1992; Mueller and Fischer, 1989). The Debenedetti European Patent Application No. 92119498.1 discloses the formation of protein microparticles using antisolvent precipitation. Schmitt (PCT publication WO 90/03782) discloses the use of antisolvent precipitation for the formation of finely divided solid crystalline powders. Hanna and York (U.S. Pat. No. 6,063,138) also disclose a method and apparatus for the formation of particles of given substances using supercritical fluids.

While much research has been performed, SAS can still only be used to produce particles in the 1-10 μm range. Therefore, attempts at adjusting the SAS process have been made in order to address this issue. For example, the use of a coaxial nozzle (PCT publication WO 95/01221) was employed to co-introduce the supercritical fluid and solution, allowing for better atomization of the solution jet. Randolph et al disclose in U.S. Pat. Nos. 5,833,891 and 5,874,029 use of an ultrasonic nozzle. Gupta et al expanded the technique in U.S. Pat. No. 6,620,351 by employing a vibrating surface in order to atomize the jet into microdroplets and provide a narrow size distribution.

### SUMMARY OF THE INVENTION

The present invention provides a method for manufacturing very small particles of anticancer molecules and poorly water soluble molecules comprising the following: providing a contained space, applying a solution having at least a solvent and the anticancer molecules on or close to a surface vibrating at a desired frequency within the contained space, applying a compressed antisolvent to the contained space, and choosing the antisolvent such that it is reasonably miscible with the solvent and that it does not dissolve the molecule substantially. The compressed antisolvent is near or above its critical point and in the liquid state. The size of the particles can be changed by changing the amplitude or frequency of vibration. The frequency can be varied from 10 Hz to 1 Ghz but is preferably in the range of 0.5 kHz and 0.5 GHz. The pressure and temperature of the contained space can be controlled and the temperature can be varied between 0.1T_{c} and 5T_{c}. The application of the solution and antisolvent is continuous as well as the collection of the particles. The solvent and antisolvent are both selected from the group consisting of ethanol, methanol, hexane, pentanes, dichloromethane, heptanes, carbon dioxide, ethane, propane, butane, sulfur hexafluoride, fluoroform, chloroform, isobutane, tetrahydrofuran, 1methyl-2-pyrrolidone, dimethyl sulfoxide, dimethyl formamide, dimethyl acetamide and a combination thereof. However, the preferred antisolvent is carbon dioxide.

The present invention also provides for a pharmaceutical composition comprising particles manufactured according to the aforementioned method and at least one stabilizer. The present invention also provides for an intravenous administration composition comprising particles manufactured according to the aforementioned method and at least one stabilizer. The composition further comprises at least one isotonic liquid carrier. This carrier is either saline or dextran. Stabilizers are selected from the group consisting of polysorbate-80, pluronic block copolymers, lecithin, polyethylene glycol, dextran and a combination thereof. The particles are collected inside the contained space in a liquid medium where the liquid medium is aqueous, organic and substantially nonsolvent for the anticancer molecules, or organic with small dissolving power for the anticancer molecules. The liquid medium may also be an isotonic carrier and contain one or more stabilizers. The contained space can withstand pressures close to 50,000 psi and temperatures close to 400° C. The produced solid particles are associated with a desired free energy. The produced particles may be amorphous or crystalline. Different crystal structures can result from the following factors: change in temperature, change of solvent, change of composition of solvents, change of antisolvent, change of antisolvent, change of composition of solvents, adding a mixing means, changing the extend of mixing and a combination thereof. The vibration of the surface is accomplished by a piezo-electric or magneto-restrictive means. The particles manufactured by the aforementioned method can have a particle size range from 0.01 nm to 50 microns and 0.01 nm to 0.5 microns. The anticancer molecule is also poorly water soluble.

### DETAILED DESCRIPTION

Definitions:

T_{c} refers to

Critical temperature of the substance which is used as the antisolvent. Depending on the context, it can be the critical temperature of the mixture of solvents and antisolvents also. Irrespective of the unit in which it is represented, the embodiments of the present invention

P_{c} refers to

Critical pressure of the substance which is used as the antisolvent. Depending on the context, it can be the critical pressure of the mixture of solvents and antisolvents also.

Desired free energy refers to

The desired free energy associated with any solid form. For example, amorphous solids have the highest free energy and most stable solid has the least free energy. Possible polymorphs, stable or otherwise may have free energies in the middle.

Anticancer molecule refers to

Any molecule that might have perceived or verified anticancer or antitumor activity.

Water insoluble molecule refers to

Any molecule that has poor water solubility

### Description

The present invention provides a method of designing and manufacturing poorly water soluble molecules. Such molecules could be from a wide variety of fields including, but not limited to, polymers, chemicals, pesticides, explosives, coatings, catalysts and pharmaceuticals. Furthermore, the present invention discloses a method of manufacturing very small particles of anticancer molecules.

A water insoluble molecule, including anticancer molecules or otherwise, is placed in solution. The solution is then loaded into either a pump or pump feeder. A contained space or particle precipitation vessel is pressurized with compressed antisolvent at the desired pressure and temperature. The compressed antisolvent to be used in the process includes, but is not limited to, ethanol, methanol, hexane, pentanes, dichloromethane, heptanes, carbon dioxide, ethane, propane, butane, sulfur hexafluoride, fluoroform, chloroform, hydrofluorocarbons, chlorofluorocarbons, isobutane, tetrahydrifuran, 1-methyl-2-pyrrolidone, dimethyl sulfoxide, dimethyl formamide, dimethyl acetamide, or a combination thereof. However, the preferred compressed antisolvent is carbon dioxide. The solvent to be used in the process includes, but is not limited to ethanol, methanol, hexane, pentanes, dichloromethane, heptanes, carbon dioxide, ethane, propane, butane, sulfur hexafluoride, fluoroform, chloroform, isobutane, tetrahydrifuran, 1methyl-2-pyrrolidone, dimethyl sulfoxide, dimethyl formamide, dimethyl acetamide.

Vibration of the surface is started by an external control mechanism and the temperature of the vessel is controlled by a water jacket, chiller, heater or other means. Frequency of vibration may be varied from 10 Hz to 1 Ghz. Varying either, or both, frequency and amplitude of vibration can change particle size. Pressure of the system is controlled by a back pressure regulator. A filtering element is provided to retain the produced particles in the vessel or in subsequent collection vessels to which the particles can be transported. Such transportation can be accomplished by the flow of antisolvent or by any other means.

After reaching the desired pressure, temperature and vibration level, all of which are controlled, a solution restriction is opened so that it can be applied on to or close to the vibrating surface. The vibration surface atomizes the droplets or ejects the droplets from the instantaneous film developed on the surface ultimately producing very fine droplets. The film thickness can be as small as a few nanometers to as high as a 20 centimeters. These droplets undergo antisolvent effect when exposed to the antisolvent and start precipitating or crystallizing as very small particles. The antisolvent removes the solvent and takes it to another vessel through a back pressure regulator where it can be removed from solvent and both the solvent and antisolvent can be separated, recycled, reused or discarded. The application of solution and antisolvent is continuous.

Particles are collected contained space or particle precipitation vessel. Antisolvent alone can be used to purge for a period of time to remove any solvent ladden antisolvent in the vicinity and to make sure the particles have the least amount of residual solvent.

In another embodiment particles can be collected in a collection zone that is subsequent in the process to the contained space or particle precipitation vessel. In yet another embodiment particles can be collected in both contained space or particle precipitation vessel and subsequent collection zones. Collection in any of the embodiments can be done in batch, semi-continuous or continuous mode.

In another embodiment of the current invention, a fluid can be inside the contained space or particle precipitation vessel and utilized a means of collection. Such fluids can be water based or organic solvent based and such liquids can also be polymer, natural macromolecule or other typical pharmaceutical excipient based. The fluids can be a solvent to the molecules or a nonsolvent to the molecules. Furthermore, the fluids may contain stabilizers, components to make them isotonic and other components that may be needed so that a final composition can be delivered to the body as a medicament.

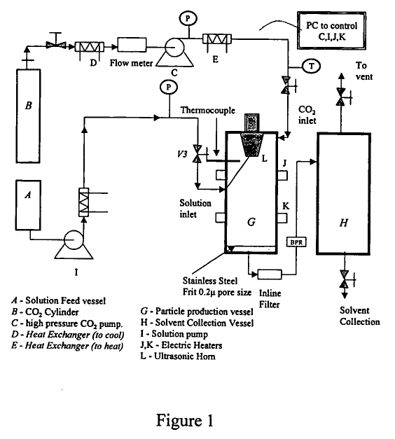

FIG. 1 illustrates an embodiment of the present invention for designing and manufacturing poorly water soluble molecules.

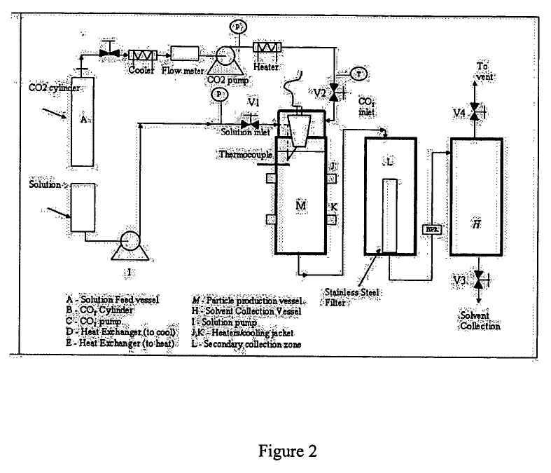

FIG. 2 also illustrates an embodiment that may also be utilized for the manufacturing very small particles of anticancer molecules. A secondary vessel was used to collect the particles at two different places. A third vessel was used to collect the solvent when the CO₂ was depressurized. This is described in FIG. 2.

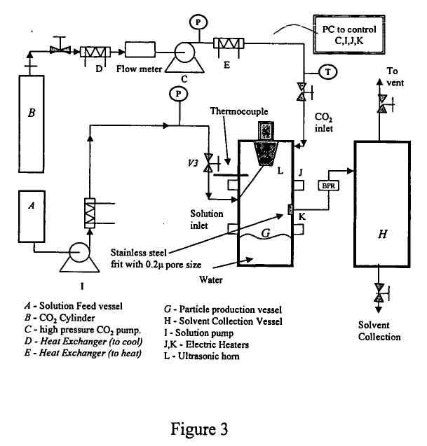

FIG. 3 illustrates another embodiment of the present invention where liquid collection can be utilized.

Particles were characterized through several methods. Scanning electron microscope imaging provided the morphology and size information. X-ray diffraction measurements revealed that the produced-particles were highly crystalline in nature. Further characterization using laser diffraction and dynamic light scattering (Photon correlation spectroscopy) provided size distribution information.

The produced particles may be made into a pharmaceutical composition by stabilizing them in an isotonic suspension.

In another embodiment, the fluids may contain stabilizers, components to make them isotonic and other components. The addition of these stabilizers and components in the fluid provides the elements needed for a composition of the particles, stabilizer(s) and component(s) that can be delivered to a human, animal or other organism as a medicament. The final composition could be a solution or a dispersion. The administration of the composition could be done through intravenous, intramuscular, interperitonial, subcutaneous, inhalation or by any other administration means.

In another embodiment, particles from any of the collection methods used in the present invention may be added to stabilizers, components to make them isotonic and other components to provide the elements needed for a composition as a medicament for delivery to a human, animal or other organism. The final composition could be a solution or a dispersion. The administration of the composition could be done intravenously or by any other method utilizing injection.

The following examples clearly illustrate the present invention:

Solutions of paclitaxel in methanol and ethanol are used in the present invention. Carbon dioxide is used as the antisolvent. The following table summarizes the conditions used for paclitaxel nanoparticle formation studies. This table provides a design with pressure and temperature maintained at 75 bar and 35° C. It was inferred from phase behavior studies that a pressure below 100 bar and temperature around 35° C. would be an optimal condition for maximum yield of particles.

TABLE 1
Experimental conditions explored for the paclitaxel with various
solvents (ethanol, methanol) with frequency at 20 kHz and 40 kHz

| | | | | | | | | Vibration Amplitude |
|---|---|---|---|---|---|---|---|---|
| | | | | | | Solution | Purge Time With | measured in terms |
| Pressure | Capillary | Temperature | Conc | CO2 Flow | Sol Flow | Injection | antisolvent, CO2 | of power input |
| bar | micron | ° C. | mg/mL | g/min | mL/min | Time, min | min | Watts |
| 75 | 100 | 35 | 30 | 50 | 0.5 | 30 | 60 | 0 |
| 75 | 100 | 35 | 30 | 50 | 0.5 | 30 | 60 | 200 |
| 75 | 100 | 35 | 30 | 50 | 2 | 15 | 60 | 200 |
| 75 | 100 | 35 | 5 | 50 | 0.5 | 120 | 60 | 200 |
| 75 | 100 | 35 | 5 | 50 | 2 | 45 | 60 | 200 |
| 75 | 100 | 35 | 5 | 50 | 0.5 | 88 | 60 | 0 |
| 75 | 100 | 35 | 5 | 50 | 2 | 37 | 60 | 0 |
| 75 | 100 | 35 | 17.5 | 50 | 1.25 | 30 | 60 | 100 |
| 75 | 100 | 35 | 17.5 | 50 | 1.25 | 30 | 60 | 100 |
| 75 | 100 | 35 | 30 | 50 | 0.5 | 20 | 60 | 0 |
| 75 | 100 | 35 | 30 | 50 | 0.5 | 30 | 60 | 200 |
| 75 | 100 | 35 | 30 | 50 | 2 | 15 | 60 | 0 |
| 75 | 100 | 35 | 30 | 50 | 2 | 15 | 60 | 200 |
| 75 | 100 | 35 | 5 | 50 | 2 | 45 | 60 | 200 |
| 75 | 100 | 35 | 5 | 50 | 0.5 | 120 | 60 | 0 |
| 75 | 100 | 35 | 17.5 | 50 | 1.25 | 30 | 60 | 100 |
| 75 | 100 | 35 | 17.5 | 50 | 1.25 | 30 | 60 | 100 |
| 200 | 100 | 60 | 15 | 50 | 1 | 30 | 60 | 0 |

TABLE 2
Experimental conditions explored for the camptothecin in
various solvents (dimethyl sulfoxide, dimethyl formamide)

| | | Vibration Amplitude | | | | | Solution | |
|---|---|---|---|---|---|---|---|---|
| | Solution | measured in terms | | | | | injection | Purge Time With |
| | Flow rate | of power input | Sol. Conc | CO2 Flow | T, | P | time | antisolvent, CO2 |
| Exp # | ml/min | W | mg/mL | g/min | C. | bar | min | Min |
| 1 | 2 | 0 | 5 | 50 | 35 | 75 | 30 | 120 |
| 2 | 0.5 | 200 | 5 | 50 | 35 | 75 | 120 | 120 |
| 3 | 2 | 200 | 5 | 50 | 35 | 75 | 30 | 120 |
| 4 | 2 | 0 | 5 | 50 | 60 | 75 | 30 | 120 |
| 5 | 2 | 200 | 5 | 50 | 60 | 75 | 30 | 120 |
| 6 | 0.5 | 0 | 5 | 50 | 35 | 75 | 60 | 120 |
| 7 | 0.5 | 200 | 5 | 50 | 60 | 75 | 120 | 120 |
| 8 | 0.5 | 0 | 5 | 50 | 60 | 75 | 60 | 120 |
| 9 | 1.25 | 100 | 5 | 50 | 47.5 | 75 | 45 | 120 |
| 10 | 1.25 | 100 | 5 | 50 | 47.5 | 75 | 45 | 120 |

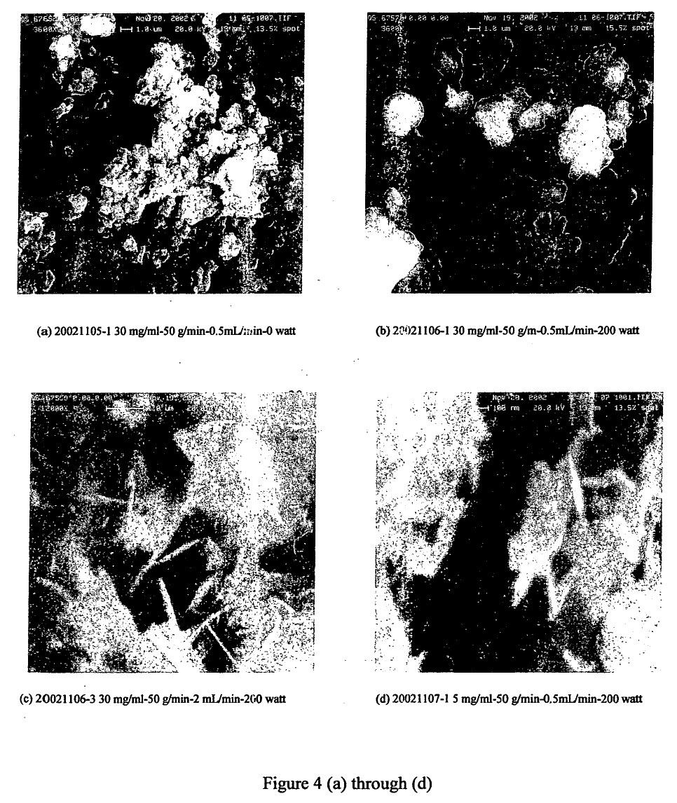

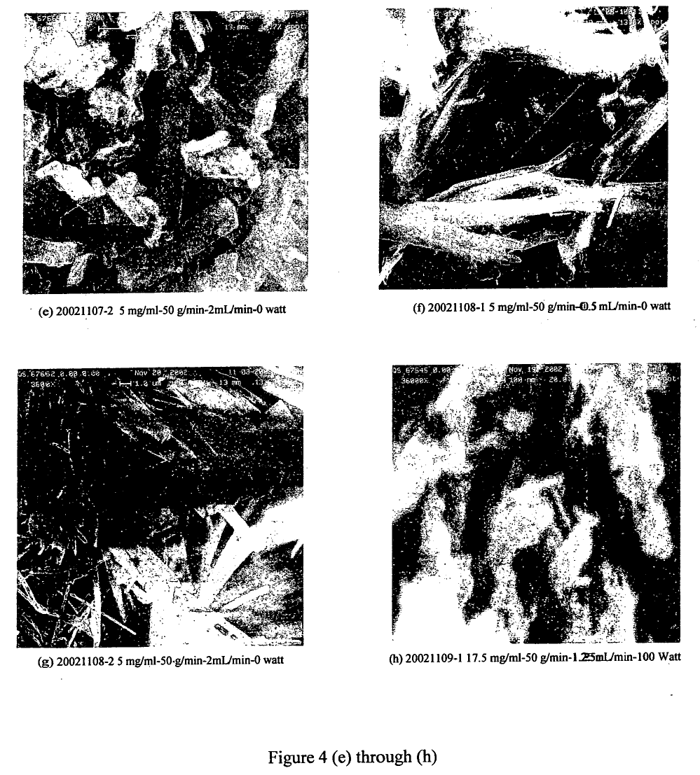

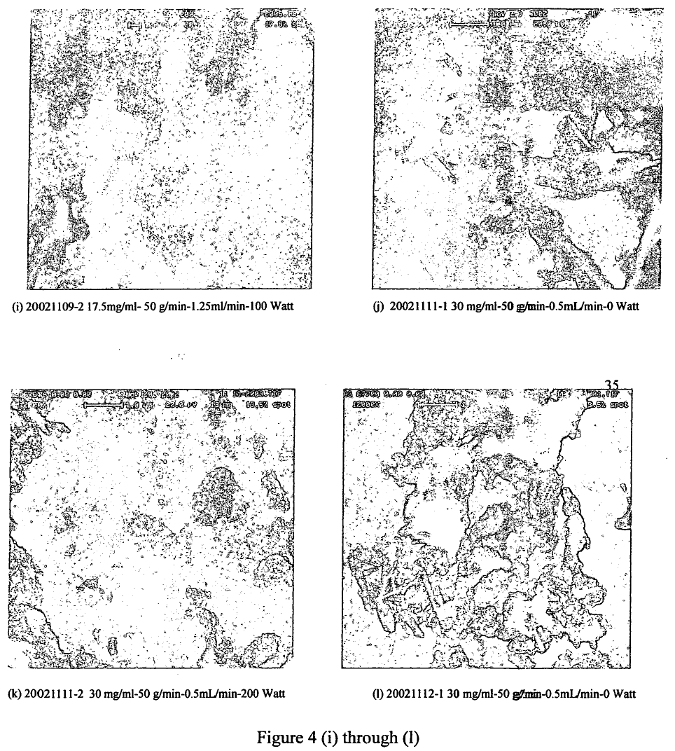

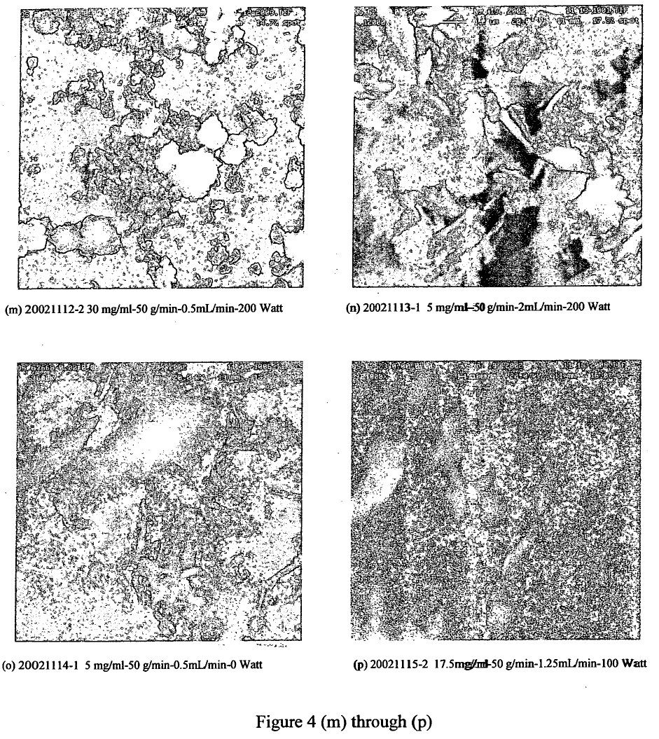

Scanning electron microscope pictures in FIG. 4 provide information about particle size and morphology information. The captions at the bottom of each micrograph list the conditions and can also be interpreted using the table above.

In addition to the particle size distribution measurements, x-ray diffraction patterns of the produced powder were measured. A portion of each sample was back-loaded into an XRD holder for analysis. The samples were run on a Philips XRD unit from 4.0 to 34° 2θ at 1.0°/min with a step size of 0.05° using graphite monochromatized copper radiation. The following graph summarizes the XRD patterns of the samples.

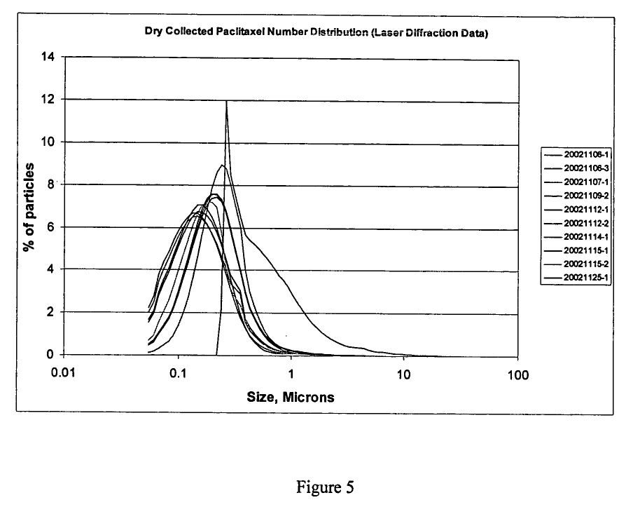

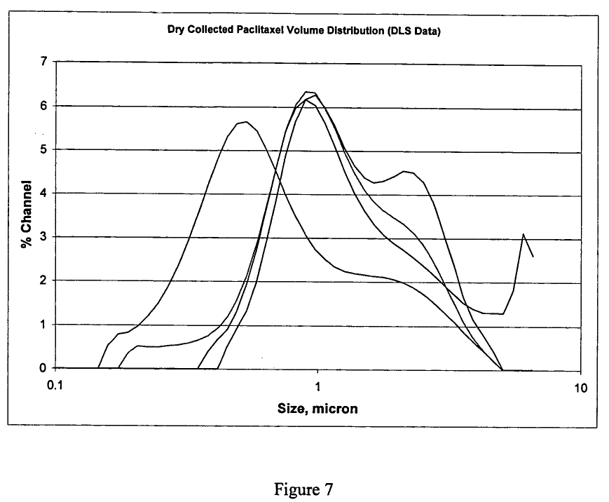

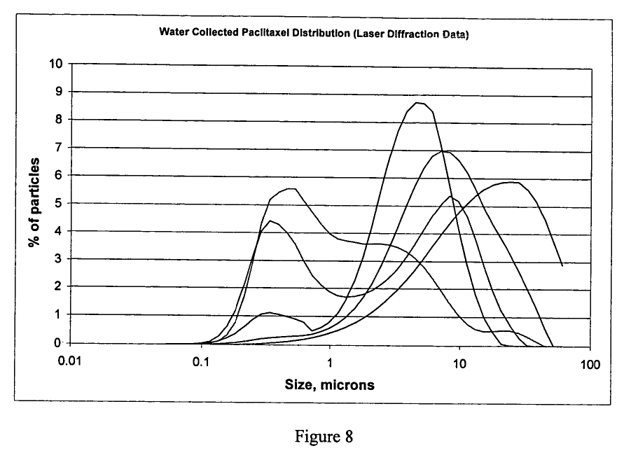

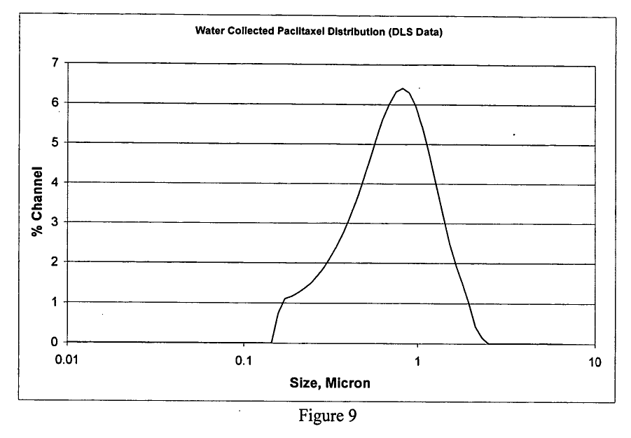

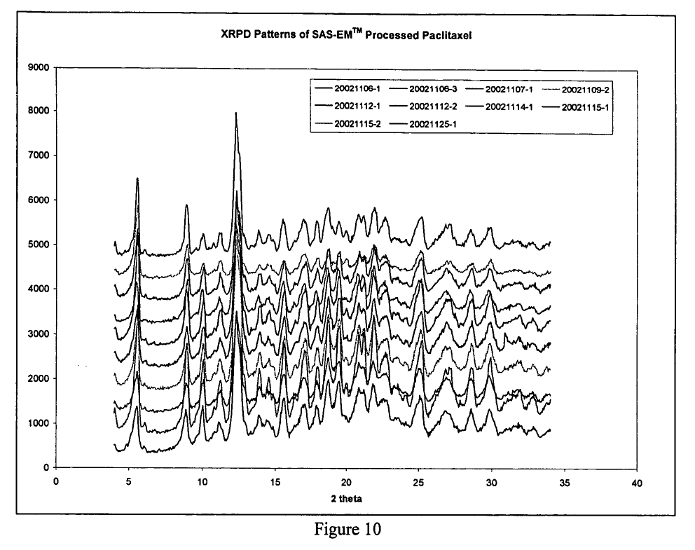

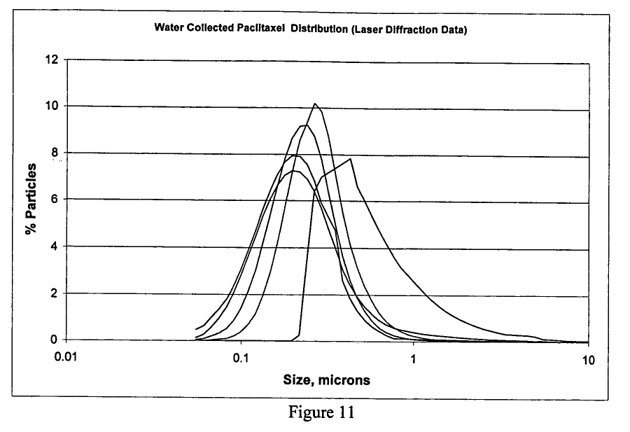

Further characterization of the particle size distribution through light scattering techniques provided the following information. Selective results are summarized in FIGS. 5 through 11 with appropriate sample names.

The following tables show additional experiments that were performed in order to demonstrate the present invention.

TABLE 3
Experimental conditions used in producing the particles as
per the current invention using dichloromethane as solvent

| | | | | | | | | Antisolvent |
|---|---|---|---|---|---|---|---|---|
| | | | | | | | Sol. Flow | (CO₂) purge |
| | T | P | Sol. Flow | CO₂ Flow | Vibration | Sol. Conc | time | time |
| Row # | C. | bar | ml/min | g/min | watts | mg/ml | min | min |
| 1 | 35 | 75 | 0.5 | 100 | 0 | 20 | 40 | 60 |
| 2 | 35 | 75 | 0.5 | 100 | 200 | 20 | 40 | 60 |
| 3 | 35 | 75 | 2 | 100 | 0 | 20 | 10 | 60 |
| 4 | 35 | 75 | 2 | 100 | 200 | 20 | 10 | 60 |
| 5 | 70 | 75 | 0.5 | 100 | 0 | 20 | 40 | 60 |
| 6 | 70 | 75 | 0.5 | 100 | 200 | 20 | 40 | 60 |
| 7 | 70 | 75 | 2 | 100 | 0 | 20 | 10 | 60 |
| 8 | 70 | 75 | 2 | 100 | 200 | 20 | 10 | 60 |
| 9 | 52.5 | 75 | 1.25 | 100 | 100 | 20 | 16 | 60 |
| 10 | 35 | 75 | 1.25 | 100 | 100 | 20 | 16 | 60 |
| 11 | 70 | 75 | 1.25 | 100 | 100 | 20 | 16 | 60 |
| 12 | 52.5 | 75 | 0.5 | 100 | 100 | 20 | 40 | 60 |
| 13 | 52.5 | 75 | 2 | 100 | 100 | 20 | 10 | 60 |
| 14 | 52.5 | 75 | 1.25 | 100 | 0 | 20 | 16 | 60 |
| 15 | 52.5 | 75 | 1.25 | 100 | 200 | 20 | 16 | 60 |

TABLE 4
Experimental conditions used in producing the particles as per the current invention using dichloromethane as solvent

| | | | | | | | Solution injection | Vibration amplitude |
|---|---|---|---|---|---|---|---|---|
| | | T Or | | | | | time/Antisolvent | Measured as power |
| P | Capillary | T range | Conc | CO2 Flow | Sol Flow | | Purge Time | input |
| bar | micron | C. | mg/mL | g/min | mL/min | Solvent | min | Watts |
| 100 | 40 | 35 | 40 | 50 | 0.5 | Methylene Chloride | 15/60 | 0 |
| 75 | 100 | 35 | 40 | 100 | 2 | Methylene Chloride | 15/60 | 100 |
| 75 | 100 | 52.5 | 40 | 100 | 1.25 | Methylene Chloride | 22190 | 100 |
| 75 | 100 | 35 | 20 | 100 | 0.5 | Methylene Chloride | 40/60 | 0 |
| 75 | 100 | 35 | 20 | 100 | 0.5 | Methylene Chloride | 40/60 | 200 |
| 75 | 100 | 35 | 20 | 100 | 2 | Methylene Chloride | 10/60 | 0 |
| 75 | 100 | 35 | 20 | 100 | 2 | Methylene Chloride | 10/60 | 200 |
| 75 | 100 | 70 | 20 | 100 | 0.5 | Methylene Chloride | 40/60 | 0 |
| 75 | 100 | 70 | 20 | 100 | 2 | Methylene Chloride | 10/60 | 0 |
| 75 | 100 | 70 | 20 | 100 | 2 | Methylene Chloride | 10/60 | 200 |
| 75 | 100 | 46/58 | 20 | 100 | 1.25 | Methylene Chloride | 16/60 | 100 |
| 75 | 100 | 30/36 | 20 | 100 | 1.25 | Methylene Chloride | 16/60 | 100 |
| 75 | 100 | 44/55 | 20 | 100 | 0.5 | Methylene Chloride | 40/60 | 100 |
| 75 | 100 | 44/56 | 20 | 100 | 2 | Methylene Chloride | 10/60 | 100 |

We claim:

1. A method for manufacturing very small particles of anticancer molecules comprising:
a. Providing a contained space
b. applying a solution having at least a solvent and the anticancer molecules on or close to a surface vibrating at a desired frequency within the contained space; and
c. applying a compressed antisolvent to the contained space; and
d. choosing the antisolvent such that it is reasonably miscible with the solvent and antisolvent does not dissolve the molecule substantially.

2. A method for manufacturing very small particles of poorly water soluble molecules comprising:
a. Providing a contained space
b. applying a solution having at least a solvent and the anticancer molecules on or close to a surface vibrating at a desired frequency within the contained space; and
c. applying a compressed antisolvent to the contained space; and
d. choosing the antisolvent such that it is reasonably miscible with the solvent and the antisolvent does not dissolve the molecule substantially.

3. The method as in claim 1 or claim 2 wherein the compressed antisolvent is near its critical point.

4. The method as in claim 1 or claim 2 wherein the compressed antisolvent is above its critical point

5. The method as in claim 1 or claim 2 wherein the compressed antisolvent is in liquid state.

6. The method as in claim 1 or claim 2 wherein the particle size can be changed by changing the amplitude of vibration

7. The method as in claim 1 or claim 2 wherein the particle size can be changed by changing the frequency of vibration

8. The method as in claim 1 or claim 2 wherein the frequency can be varied from 10 Hz to 1 Ghz.

9. The method as in claim 1 or claim 2 wherein the frequency is preferably between 0.5 kHz and 0.5 Ghz.

10. The method as in claim 1 or claim 2 wherein the temperature of the contained space can be controlled

11. The method as in claim 1 or claim 2 wherein the pressure of the contained space can be controlled.

12. The method as in claim 1 or claim 2 wherein the temperature of the contained space can be varied between 0.1 times T_{c} and 5 times T_{c}

13. The method as in claim 1 or claim 2 wherein the application of solution is continuous

14. The method as in claim 1 or claim 2 wherein the application of antisolvent is continuous

15. The method as in claim 1 or claim 2 wherein the antisolvent is selected from the group consisting of ethanol, methanol, hexane, pentanes, dichloromethane, heptanes, carbon dioxide, ethane, propane, butane, sulfur hexafluoride, fluoroform, chloroform, hydrofluorocarbons, chlorofluorocarbons, isobutane, tetrahydrofuran, 1-methyl-2-pyrrolidone, dimethyl sulfoxide, dimethyl formamide, dimethyl acetamide and a combination thereof.

16. The method as in claim 1 or claim 2 wherein the antisolvent is carbon dioxide

17. The method as in claim 1 or claim 2 wherein the solvent is selected from the group consisting of ethanol, methanol, hexane, pentanes, dichloromethane, heptanes, carbon dioxide, ethane, propane, butane, sulfur hexafluoride, fluoroform, chloroform, isobutane, tetrahydrofuran, 1methyl-2-pyrrolidone, dimethyl sulfoxide, dimethyl formamide, dimethyl acetamide and a combination thereof.

18. The method as in claim 1 or claim 2 wherein the collection of the particles is continuous

19. A pharmaceutical composition comprising
a. Particles manufactured according to claim 1 or claim 2; and
b. At least one stabilizer.

20. An intravenous administration composition comprising
c. Particles manufactured according to claim 1 or claim 2; and
d. At least one stabilizer.

21. The composition as in 20 further comprising at least one isotonic liquid carrier.

22. The formulation as in claim 1 or claim 20 wherein the stabilizers are selected from the group consisting of polysorbate-80, pluronic block copolymers, lecithin, polyethylene glycol, dextran and a combination thereof.

23. The method as in claim 1 or claim 21 wherein the isotonic liquid carrier is saline or dextran.

24. The method as in claim 1 or claim 2 wherein the particles are collected inside the contained space in a liquid medium

25. The method as in claim 1 or claim 24 wherein the liquid medium is aqueous

26. The method as in claim 1 or claim 24 wherein the liquid medium is organic and substantially nonsolvent for the anticancer molecules

27. The method in claim 1 or claim 24 wherein the liquid medium is organic and has a small dissolving power for the anticancer molecules

28. The method as in claim 1 or claim 24 wherein the liquid medium is an isotonic carrier

29. The method as in claim 1 or claim 24 wherein the liquid medium contains one or more stabilizers

30. The method as in claim 1 or claim 2 wherein the contained space can withstand pressures close to 50,000 psi

31. The method as in claim 1 or claim 2 wherein the contained space can withstand temperatures close to 400° C.

32. The method as in any of the above claims wherein the produced solid particles are associated with a desired free energy.

33. The method as in any of the above claims wherein the produced particles are amorphous

34. The method as in any of the above claims wherein the produced particles are crystalline

35. The method as in any of the above claims wherein a factor selected from the group consisting of change in temperature, change of solvent, change of composition of solvents, change of antisolvent, change of antisolvent, change of composition of solvents, adding a mixing means, changing the extend of mixing and a combination thereof result different crystal structures.

36. Methods and particles as in any one of the above claims wherein the vibration of the surface is accomplished by a piezo-electric or magneto-restrictive means

37. Particles manufactured by any of the above claims wherein the particle size range is from 0.01 nm to 50 microns

38. Particles manufactured by any of the above claims wherein the particle size range is from 0.01 nm to 0.5 microns

39. Methods and particles as in any one of the above claims wherein the anticancer molecule is poorly water soluble
